(19) United States
(12) Patent Application Publication
(10) Pub. No.: US 2026/0000878 A1
(43) Pub. Date: Jan. 1, 2026

(54) ACOUSTOFLUIDIC PATCH FOR SAMPLING AND DELIVERY

(71) Applicant: The Trustees of Indiana University, Bloomington, IN (US)

(72) Inventors: Feng GUO, Bloomington, IN (US); Hongwei CAI, Bloomington, IN (US); Ken MACKIE, Bloomington, IN (US)

(21) Appl. No.: 19/252,811

(22) Filed: Jun. 27, 2025

Related U.S. Application Data
(60) Provisional application No. 63/665,680, filed on Jun. 28, 2024.

(51) Int. Cl. A61M 37/00; A61B 10/00

(52) U.S. Cl. CPC A61M 37/0015; A61B 10/0045; A61M 2037/0023; A61M 2037/003; A61M 2202/0007; A61M 2202/04; A61M 2205/0294; A61M 2205/3334; A61M 2210/04

(57) ABSTRACT

Disclosed herein are acoustofluidic transdermal drug delivery devices, systems, and methods for delivering a therapeutic agent to a subject or sampling a biofluid, such as interstitial fluid or blood, from the body of the subject. The disclosed device, systems, and methods involve application of an AC signal, such as radiofrequency, which generates surface acoustic waves (SAWs) upon interacting with the device, thereby inducing a streaming flow capable of enabling transdermal drug delivery or transdermal sampling of a biofluid.

### CROSS REFERENCE TO RELATED APPLICATIONS

This application claims the benefit under 35 U.S.C. § 119(e) of U.S. Provisional Application No. 63/665,680 entitled “ACOUSTOFLUIDIC PATCH FOR SAMPLING AND DELIVERY”, filed on Jun. 28, 2024, which is incorporated by reference in its entirety.

### STATEMENT OF GOVERNMENTAL RIGHTS

This invention was made with government support under DA056242 awarded by National Institutes of Health. The Government has certain rights in the invention.

### FIELD OF THE INVENTION

The present disclosure relates generally to acoustofluidic patches, transdermal patch devices, and methods of use. The disclosed acoustofluidic patches and delivery systems being capable of delivery of a desired amount of a therapeutic agent to a subject over the desired time interval. The disclosed acoustofluidic patches and systems also being capable of use for on-demand sampling of biofluids, e.g., skin interstitial fluids and blood, from the body of a subject.

### BACKGROUND

Personalized diagnosis and therapies aim to detect and treat the onset of disease in an individual patient in a convenient and real-time manner. However, challenges remain in sampling and detection of biomarkers for personalized diagnosis as well as on-demand delivery of therapeutics for personalized therapy. For example, conventional drug delivery patches only achieve the passive trans-dermal delivery of therapeutics. Accordingly, there is a need for programmable biological sampling and delivery of therapeutics, as well as precise volume control over the same. The present disclosure relates to systems, devices, and methods to meet such needs.

### SUMMARY OF THE INVENTION

A first aspect of the invention includes acoustofluidic patch devices including with components that are capable of generating a surface acoustic wave to deliver an agent or sample of a biofluid, such as surface acoustic wave (SAW) device, an acoustofluidic streaming chamber; and a hollow microneedle.

A second aspect of the invention is the acoustofluidic patch designed and used for sampling of biofluids from a subject.

A third aspect of the invention is the acoustofluidic patch designed and used for delivery of therapeutics to a subject.

In a first embodiment an acoustofluidic patch comprising a surface acoustic wave (SAW) device, an acoustofluidic streaming chamber and a hollow microneedle.

In a second embodiment, the SAW device comprises a piezoelectric substrate and at least one pair of interdigital transducers.

In a third embodiment, the acoustofluidic streaming chamber comprises an inlet, and the hollow microneedle is separably coupled to the inlet.

In a fourth embodiment, the SAW device is separatable from the acoustofluidic chamber.

In a fifth embodiment, the hollow microneedle has a tip size ranging from about 0.1 mm to about 4 mm.

In a sixth embodiment, the orientation angle of the acoustofluidic streaming chamber is 22 to 202 degrees.

In a seventh embodiment, a transdermal delivery patch wherein a fluid medium is contained within the acoustic streaming chamber and the fluid has a viscosity from about 0.5 to about 500 cp/mPa's.

In an eighth embodiment, the transdermal delivery patch wherein the fluid within the acoustofluidic streaming chamber comprises a therapeutic agent.

In a ninth embodiment, a method for transdermally administering a therapeutic agent to a subject, the method comprising applying the patch of claim 7 to the skin of the subject wherein the hollow microneedle penetrates the subject's skin, activating the SAW device through actuation of an AC signal to generate an acoustic stream within the acoustofluidic chamber and move fluid from the acoustofluidic chamber out the hollow microneedle.

In a tenth embodiment, the method for administering a therapeutic agent wherein the AC signal comprises a radio frequency signal.

In an eleventh embodiment the actuating of the AC signal occurs for at least 30 seconds, 1 min, 5 min, 10 min, 15 min, 20 min, 25 min, 30 min, 35 min, 40 min, 45 min, 50 min, 55 min, or 60 min.

In a twelfth embodiment, the therapeutic agent has an average flow rate of about 5-60 μL/min, 5-40 μL/min, 5-20 μL/min, 10-50 μL/min, 10-15 μL/min, 15-25 μL/min, 15-55 μL/min, 25-55 μL/min through the hollow microneedle.

In a thirteenth embodiment, a biofluid sampling patch comprising the acoustofluidic patch of claim 1 wherein the acoustofluidic streaming chamber contains no fluid prior to use.

In a fourteenth embodiment, sampling patch further comprising an organic electrochemical transistor.

In a fifteenth embodiment, a method for transdermal sampling a biofluid from a subject, the method comprising applying the patch of claim 12 to the skin of the subject wherein the hollow microneedle penetrates the subject's skin, activating the SAW device through actuating an AC signal to generate an acoustic stream within the acoustofluidic chamber and moving fluid into the acoustofluidic chamber through the hollow microneedle.

In a sixteenth embodiment, the biofluid is blood, interstitial fluid, or a combination thereof.

In a seventeenth embodiment, the sampling of the biofluid comprises removing a biofluid sample volume from the subject of 0.1-5 μL, 0.1-1 μL, 0.1-0.75 μL, 0.1-0.5 μL, 0.1-0.25 μL, 0.2-0.6 μL, or 0.25-0.5 μL, wherein each range is inclusive.

In an eighteenth embodiment the biofluid has a flow rate of 0.5-40 μL/min, 10-20 μL/min, 10-15 μL/min, or 15-20 μL/min from the subject through the hollow needle, the biofluid having a temperature of about 37.5° C.

In a nineteenth embodiment, a system comprising the acoustofluidic patch of claim 1, and an AC signal source, wherein upon actuation of the AC signal source, the SAW device creates an acoustic wave within the acoustofluidic streaming chamber of the patch.

### SUMMARY OF THE INVENTION

The features of the present disclosure according to some examples or embodiments will now be described, purely by way of example, with reference to the accompanying drawings, in which:

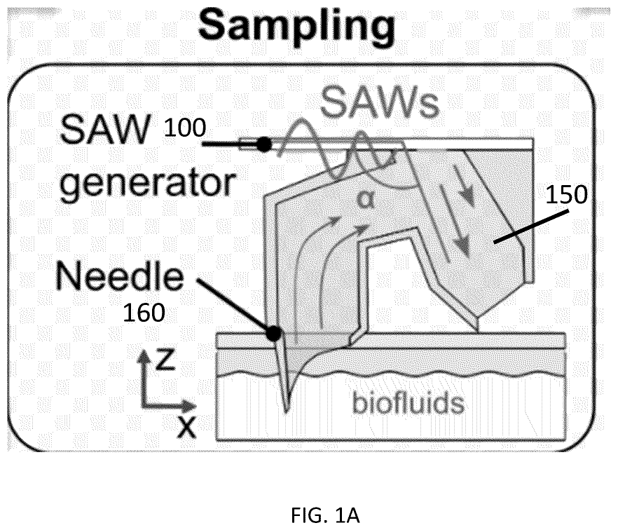

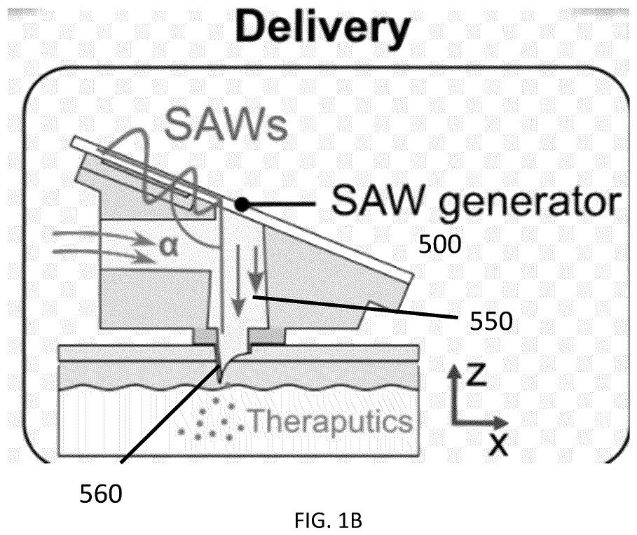

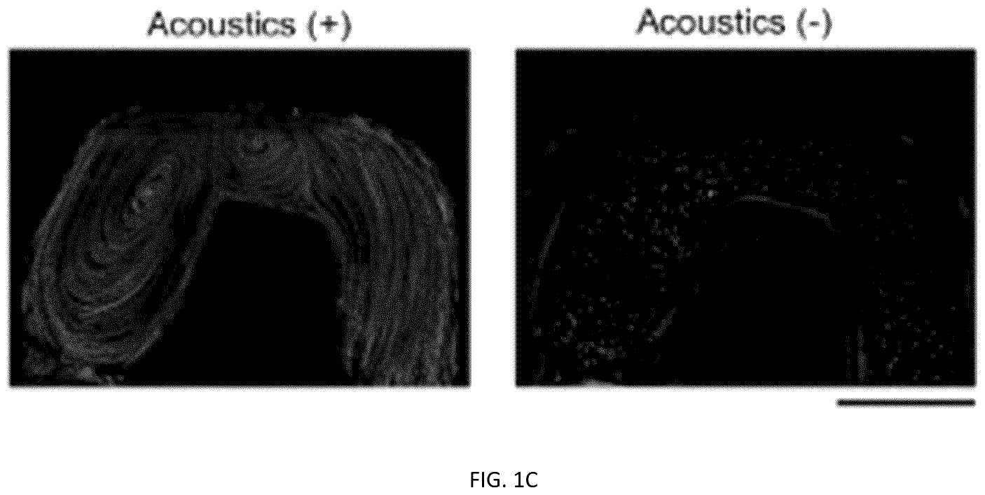

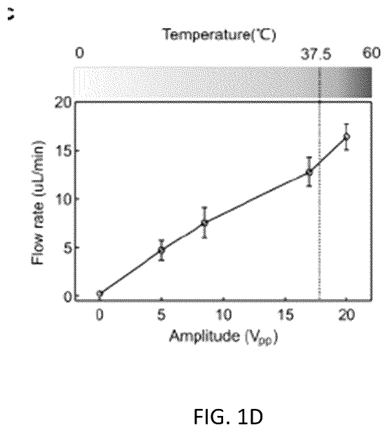

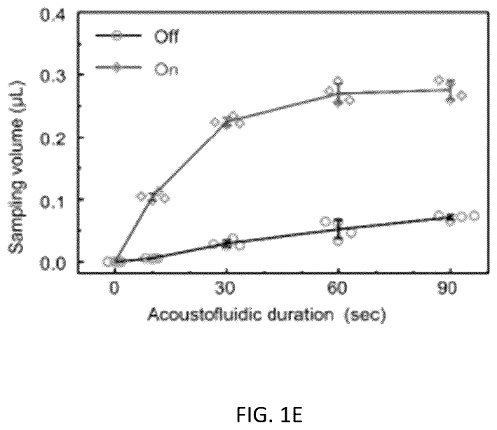

FIG. 1A is a side view illustration of an acoustofluidic patch of the present disclosure for biofluid sampling.

FIG. 1B is a side view illustration of an acoustofluidic patch of the present disclosure for fluid delivery.

FIG. 1C are images of the fluid movement within the acoustic streaming chamber

FIG. 1D is a graph illustrating the flow rate (μL/min) and temperature at different different peak-to-peak input voltage (Vpp).

FIG. 1E is a graph illustrating the sampling volume (μL) over time (sec) with the acoustic patch on and off (acoustic duration).

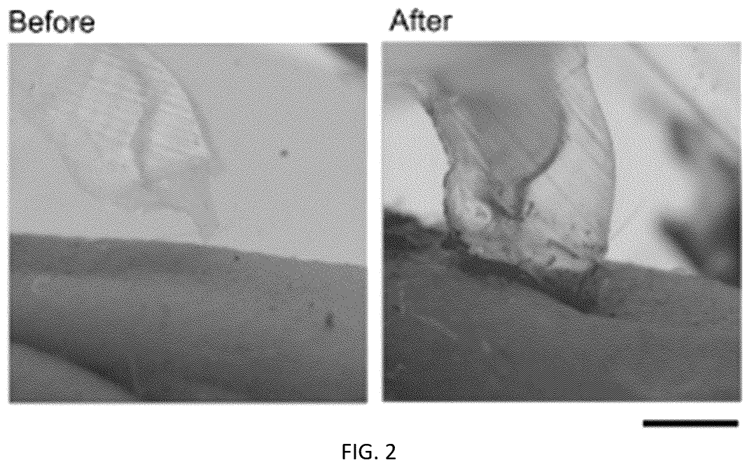

FIG. 2 are images of an acoustofluidic patch of the present invention sampling skin ISF. Scale bar: 500 μm.

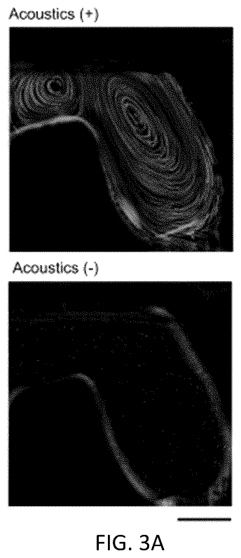

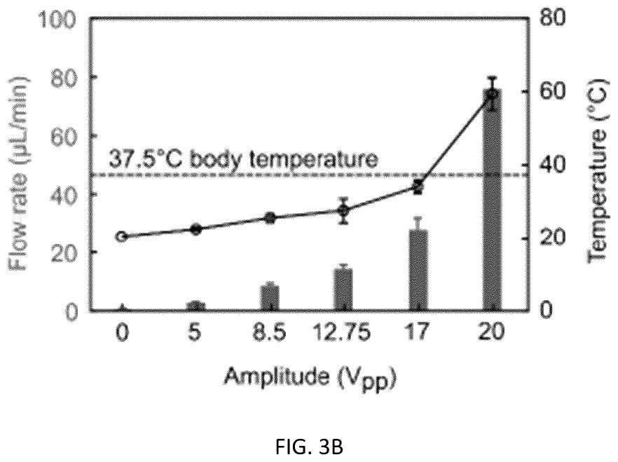

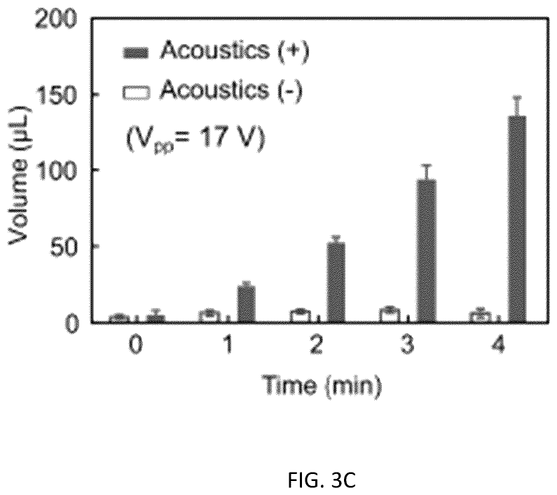

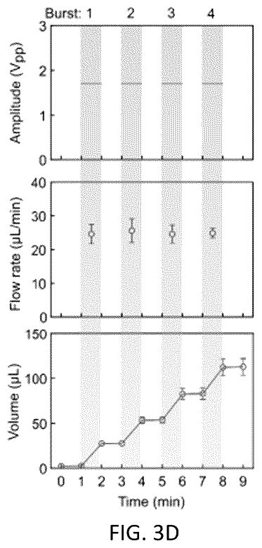

FIG. 3A are images of the fluid movement within the acoustic streaming chamber.

FIG. 3B is a graph illustrating the flow rate (μL/min) at different amplitudes (Vpp). All data presented are shown as mean±s.d., n=3-6 independent experiments.

FIG. 3C are graphs illustrating volume (μL) at different time periods of the acoustics being at an “on” state. All data presented are shown as mean±s.d., n=3-6 independent experiments.

FIG. 3D are graphs illustrating the volume (μL), flow rate (μL/min) and amplitude (Vpp) over a time course of min-burst acoustics. All data presented are shown as mean±s.d., n=3-6 independent experiments.

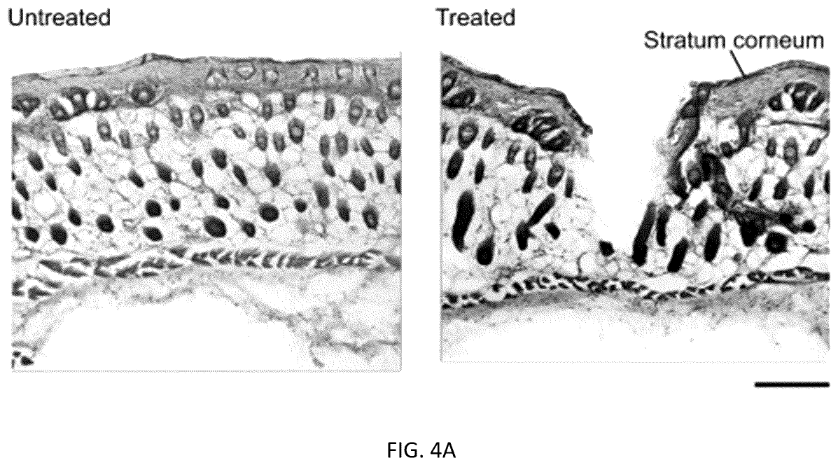

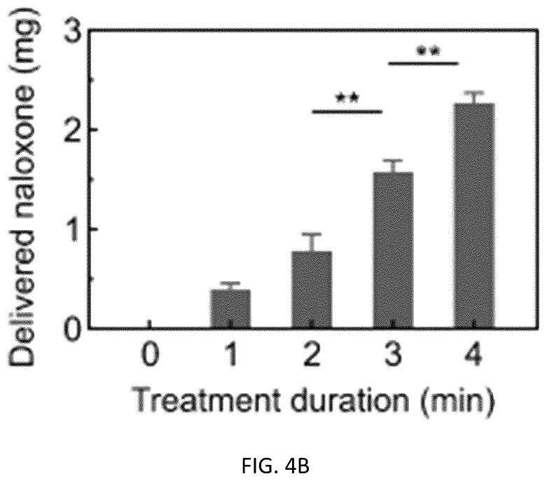

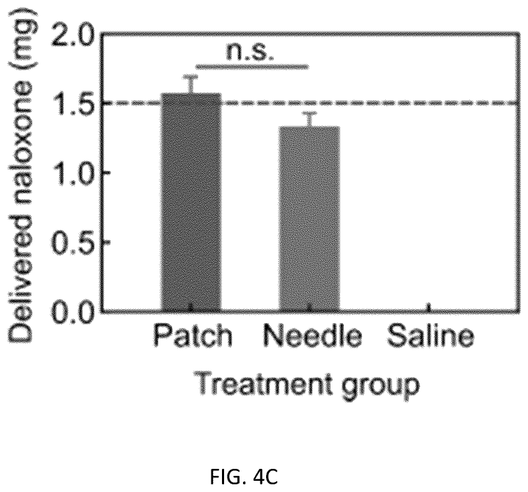

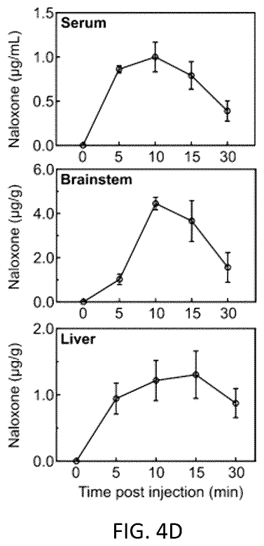

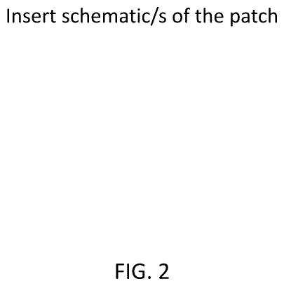

FIG. 4A are images of hematoxylin and eosin (H&E) staining of the skin cryosection with or without microneedle patch application. (Scale bar: 500 μm)

FIG. 4B is a graph showing the quantitative analysis of the naloxone transdermal delivery (delivered naloxone (mg) per treatment duration (min) with high-performance liquid chromatography-mass spectrometry (HPLC-MS). All data presented are shown as mean±s.e.m., n=3-5 independent experiments.

FIG. 4C are graphs depicting the amount of naloxone delivered via the acoustofluidic patch versus subcutaneous needle injection. All data presented are shown as mean±s.e.m., n=3-5 independent experiments.

FIG. 4D are graphs showing the serum-brain-liver distribution of naloxone after acoustofluidic patch treatment. After 3 min of patch treatment, mice were sacrificed at the indicated timepoints. Serum and tissue samples collected for naloxone measurement using HPLC-MS. All data presented are shown as mean±s.e.m., n=3-5 independent experiments.

### DETAILED DESCRIPTION

The present disclosure provides an acoustofluidic transdermal patch configured to sample fluids within the body and/or deliver drugs into the tissue. Acoustofluidic patches of the present disclosure use acoustic waves to manipulate fluidic flow in and out of the patch, thus facilitating a contact-free and biocompatible platform for biological detections or drug delivery with a small footprint. The acoustofluidic patch may comprise a surface acoustic wave (SAW) device, an acoustofluidic streaming chamber; and at least one hollow microneedle (160) positioned at the inlet of the streaming chamber. An illustration of an embodiment of the acoustofluidic transdermal patch for biofluid sampling as described herein is shown in FIG. 1A, showing the arrangement of SAW device (100), acoustofluidic streaming chamber (150), and microneedle (160). An illustration of an embodiment of the acoustofluidic patch for delivery of as described herein is show in FIG. 1B, showing the arrangement of SAW device (500), acoustofluidic streaming chamber (550), and microneedle (560). Surface acoustic waves (SAW) are created within the acoustofluidic patch to control the movement of fluids into and out of the patch through the hollow microneedle.

### Surface Acoustic Wave Device (SAW) Device

The SAW device may comprise a piezoelectric substrate and a pair of interdigital transducers. The interdigital transducers create mechanical vibrations on the surface of a piezoelectric substrate to propagate the vibration along the surface of the piezoelectric substrate as waves.

The piezoelectric substrate may be a material that exhibits the ability to generate an electric charge in response to mechanical stress. The material may also be configured to change shape or deform when an electric field is applied. Piezoelectric substrates may be made from crystals or ceramics, such as quartz, lead zirconate titanate, and zinc oxide. A suitable piezoelectric substrate may include lithium niobate.

The interdigital transducers may be a device comprising a set of electrodes on the piezoelectric substrate configured to convert electrical signals into acoustic waves and vice versa.

### Acoustofluidic Streaming Chamber

The acoustofluidic patch comprises an acoustofluidic streaming chamber. The acoustofluidic streaming chamber is positioned directly below the SAW device such that fluid within the chamber is in contact with the SAW device. The orientation angle (a) of the SAW device to the acoustofluidic streaming chamber may be 22 to 202 degrees. As shown in the FIG. 1A, for the acoustofluidic patch for sampling, the orientation angle (a) moves fluids up into the acoustofluidic streaming chamber from the microneedle. As shown in FIG. 1B, for the acoustofluidic patch for deliver, the orientation angle (a) moves fluid from the acoustofluidic streaming chamber out through the microneedle.

The acoustofluidic streaming chamber includes at least one inlet at the opposite side of the chamber from the SAW device to which the microneedle is attached. The acoustofluidic streaming chamber may also have an opening to a storage chamber, wherein the opening is positioned at the opposite end of the acoustofluidic streaming chamber from the inlet. When the acoustofluidic patch is designed for delivery (FIG. 1B), the acoustic waves push fluid out of the microneedle and pull fluid from the storage chamber into the acoustofluidic streaming chamber.

The acoustofluidic streaming chamber comprises an internal volume. Within the internal volume, a fluid medium may be present. The attenuation of the surface acoustic wave created by the SAW device within the fluid medium drives the vortex-like steady flow known as acoustic streaming. The viscosity of the fluid medium contained within the acoustofluidic patch may range from 0.5 to 500 cP/mPa·s. However, when the acoustofluidic patch is to be used as a transdermal patch for biofluid sampling, the presence of fluid within the acoustofluidic chamber is not required as fluid will flow into the chamber through the microneedle.

When the acoustofluidic patch is to be used as a transdermal patch for drug delivery to a subject, the fluid medium may comprise a therapeutic agent. Suitable therapeutic agents stored in the acoustofluidic streaming chamber may include an opioid antagonist, a benzodiazepine, an insulin composition, a sulfonylurea, a biguanide, a DPP-4 inhibitor, a GLP-1 receptor agonist, an SGLT2 inhibitor, a thiazolidinedione, a dextrose solution, diazoxide, an antihistamine, epinephrine, a coriticosteroid, an immunosuppressant, an immunomodulator, a monoclonal antibody, a nonsteroidal anti-inflammatory drug, an analgesic agent, an anti-arthritic agent, an anti-arrhythmic agent, an anti-asthmatic agent, an anaesthetic, an anticonvulsant, an antidepressant, an anxiolytic, an antibiotic, an anticancer agent, an antidiabetic agent, an antiviral agent, an anti-inflammatory agent, an antiglaucoma agent, an antiemetic, an antineoplastic, an antiparkisonian agent, an antirheumatic agent, an antipsychotic, an appetite stimulants, an appetite suppressant, an attention disorder agent, a cholesterol-lowering agent, a cardiovascular agent, central nervous system stimulant, naloxone, naltrexone, nalmefene, methylnaltrexone, buprenorphine, methadone, suboxone, clonidine, lofexidine, a derivatives thereof, or a combination thereof.

### Microneedle

The acoustofluidic sampling patch comprised at least one hollow microneedle (160). The microneedles may be coupled to one or more inlet (151) of the acoustofluidic streaming chamber (150). One of skill in the art would appreciate that the number of microneedles and pattern of microneedles may be modified for functional reasons to increase or decrease the number of points of penetration into the skin or the subject. The number and pattern of the microneedles may also be modified for comfort and/or aesthetic reasons. In one embodiment the plurality of microneedles may be positioned in a grid orientation where each microneedle in the plurality of microneedles may be equal distant from the next microneedle in the plurality of microneedles.

The hollow microneedle may have a hollow bore diameter ranging from about 0.1 to 1.0 mm, 0.4 to 0.8 mm, 0.5 to 1.0 mm, or 0.6 to 0.8 mm, where each range is inclusive. The hollow microneedle may have a hollow bore diameter of about 0.4 mm, 0.6 mm, or 0.8 mm. The bore size of the microneedle may determine the flow rate of the therapeutic agent. The therapeutic agent may have an average flow rate of about 5-60 μL/min, 5-40 μL/min, 5-20 μL/min, 10-50 μL/min, 10-15 μL/min, 15-25 μL/min, 15-55 μL/min, 25-55 μL/min through the hollow microneedle of the acoustofluidic sampling patch.

A tip of the hollow microneedle may be configured to penetrate into a subject's tissue such that the hollow microneedle is within the tissue of the subject and the acoustofluidic sampling patch rests against the surface of the tissue and the microneedle penetrates through the stratum corneum of the subject's skin. The hollow microneedle (160) may have a length ranging from about tip size ranging from about 0.1 mm to about 4 mm. The length of the microneedle will depend upon the depth of penetration into the skin desired, with microneedles of about 0.2 to about 1 mm extending through the stratum corneum into the epidermis and microneedles of about 1.5 to about 4 mm reaching into the dermis layer.

In one embodiment the SAW device may be separated from the acoustofluidic streaming chamber such that the SAW device may be reused and attached to another acoustofluidic streaming chamber.

### Acoustofluidic Therapeutic Delivery and Sampling System and Method of Use

The present disclosure may further provide a system comprising the acoustofluidic transdermal patch and a portable AC signal source positioned within a working range of the transdermal patch.

The AC signalling source may comprise an actuator. Actuating the AC signal source may include controlling the AC signal to have resonant frequency of from about 1-50 MHz, 25-40 MHz, 30 to 50 MHz, 30-40 MHz, 35-40 MHz, or 35-45 MHz, where each range is inclusive.

Actuating the AC signal may include controlling the AC signal to have an amplitude of 1-30 volts peak-to-peak (Vpp), 10-30 Vpp, 15-25 Vpp, 15-20 Vpp, or 20-25 Vpp, where each range is inclusive. The actuating of the AC signal may occurs for at least 30 seconds, 1 min, 5 min, 10 min, 15 min, 20 min, 25 min, 30 min, 35 min, 40 min, 45 min, 50 min, 55 min, 60 min 75 min, or a range using any of the foregoing values as endpoints.

The AC signal may have a duty cycle of about 25-75%, 30-60%, 40-70%, 45-65%, 45-55%, 50-65%, 50-75%, or 45-65%, where each range is inclusive.

By assembling surface acoustic waves into the patches with sharp hollow needle tips, digitalized acoustic streaming can be introduced for pumping fluids into or out of the disclosed patch device through the microchannel.

### Therapeutic Delivery

The acoustofluidic transdermal patch may be configured to be used to administer a therapeutic agent. The acoustofluidic patch is applied to the skin of a subject such that the microneedle pierces the tissue of the subject. Once the patch is applied, an alternating current may be applied initiating the SAW device to produce vibrations. The vibrations generate acoustic streaming in the acoustic streaming chamber, the acoustic streaming capable of pumping a fluid out of the acoustic streaming chamber through the hollow microneedle.

Programmable delivery of therapeutics can be achieved by pumping liquid drug into the tissue or dissolving solid drug into biofluids, thereby supplying drug to the tissue. The acoustofluidic sampling patch may deliver a variety of types of drugs, such as powders, cells, liquids, and solids, via transdermal drug delivery and/or the hollow needle tip. Once acoustic streaming is triggered, the patch devices can enable the on-demand trans-dermal delivery of therapeutics through skin tissues for regulating drug pharmacokinetics.

### Sampling

The acoustofluidic sampling patch may be configured to be used to sample biological fluids from a subject. In some embodiments, the acoustofluidic sampling patch is removed after sampling and the collected fluid is tested. In some embodiments, an organic electrochemical transistor is present in the sampling patch and may further comprise a biological sensor, such as an organic semiconductor, that detects the presence of specific elements or compounds in the collected fluid as it comes into the acoustofluidic streaming chamber.

After penetrating the sharp hollow needle tip into skin tissue (or deep tissue), on-demand sampling of biofluids, such as interstitial fluids (or bloods), blood, saliva, sweat, and skin cells with precise volume control can be achieved. This is useful for biomarker detection in the biofluids which can be used for disease diagnosis and/or monitoring treatment response to conditions such as drug overdose, hypo/hyperglycemia, food allergy, and immunological responses. The sample taken by the acoustofluidic sampling patch may be analyzed for at least one of blood sugar, drug concentration, hormone levels, immunological response, hypo/hyperglycemia, food allergy response.

The acoustofluidic sampling patch may be configured to removing a sample volume from the subject of 0.1-5 μL, 0.1-1 μL, 0.1-0.75 μL, 0.1-0.5 μL, 0.1-0.25 μL, 0.2-0.6 μL, or 0.25-0.5 μL, where each range is inclusive. The sample may flow from the subject through the hollow microneedle at a flow rate of a flow rate of 0.5-40 μL/min, 10-20 μL/min, 10-15 μL/min, or 15-20 μL/min, the biofluid having a temperature of about 37.5° C., where each range is inclusive.

This sampling is applicable to disease diagnosis and monitoring treatment and the status of the subject's body. The subject may be a human or other suitable animal.

### Example 1: Acoustofluidic Patch for Biofluid Sampling

The acoustofluidic patch. An acoustofluidic patch was prepared as illustrated in FIG. 1A. A piezoelectric substrate (LiNbO₃) substrate was fabricated with interdigital transducers (IDTs). A 3D printed acoustic streaming chamber and a hollow microneedle was attached to the SAW device with a transfer tape. Once IDT generated surface acoustic waves (SAWs) propagated along the surface and reached the liquid/solid interface, it would induce a streaming flow thus enabling the interstitial fluid (ISF) sampling through the hollow microneedle.

Movement within the acoustic streaming chamber was confirmed. As shown in FIG. 1C, when the acoustics were turned on, the streamline within the chamber was visualized with 2 μm microbeads as tracers. FIG. 1D shows the flow rate and device temperature at different peak-to-peak input voltage (Vpp) and it is quantified by the multiply of the velocity of the tracer entering the streaming chamber.

Biofluid sampling was confirmed. FIG. 1E shows the sampling volume achieved over time with the acoustic patch on and off. Images of the sampling are shown in FIG. 2. The acoustofluidic flow significantly increased the volume delivered when passive diffusion became the driving force.

### Example 2: Acoustofluidic Patch for Drug Delivery

The acoustofluidic patch. The acoustofluidic patch was prepared as illustrated in FIG. 1A. The acoustofluidic patch comprised a 3D-printed acoustofluidic streaming chamber integrated with a hollow microneedle and a surface acoustic wave (SAW) device. The SAW device was fabricated by patterning interdigital transducers (IDTs) on the piezoelectric substrate lithium niobate (LiNbO₃). When the IDTs are excited by a radio frequency (RF) signal, the vibrations of the substrate generate a SAW propagating along the surface. To take advantage of the maximum streaming velocity, the chamber was aligned along the Rayleigh angle, i.e., the specific angle for the SAW energy leaking into the fluid. By operating at resonant frequency (37.4 MHz) with an amplitude of 17 Vpp and 50% duty cycle, our acoustofluidic patch achieved an average flow rate of 28±2.3 μL/min.

Movement within the acoustofluidic streaming chamber was confirmed. As shown in FIG. 3A, the acoustics-induced microfluidic flow (i.e., the acoustic streaming) was visualized with 2 μm fluorescence beads as tracers. Acoustics (+) acoustics (−) indicate the “on” and “off” states of surface acoustic wave (SAW), respectively, with an optimized frequency of 37.4 MHz and a duty cycle of 50%. When acoustics were on, the streamline was shown by overlaying 25 frames of the fluorescence imaging taken at 50 frames per second. (Scale bar: 1 mm). Upon reaching the solid-liquid interface of the streaming chamber, SAW decays exponentially thus inducing a vortex-like, non-oscillatory steady fluid flow known as acoustic streaming.

Flow rate. The flow rate induced by the acoustic streaming is amplitude dependent (FIG. 3B). Additionally, the on-chip heating was maintained around body temperature (37.5° C.) when the device was running at 17 Vpp. The acoustic-induced heating was monitored with an infrared temperature sensor.

Delivery volume and control of dosing. The delivery volume was measured with or without acoustofluidics (acoustics (+) and (acoustics (−), FIG. 3C). The experimental results showed that targeted delivery volume could be precisely controlled by acoustic duration, which is significantly greater than passive diffusion (acoustic (−)). Moreover, this enables programmable delivery through multi-bursting over time (each burst being 17 Vpp for 3 min, 50% duty cycle). The delivered volume can be adjusted by manipulating the acoustic duration (acoustics (+)), and delivery by acoustic streaming is far more efficient than passive diffusion (acoustics (−)). This tunability of the patch allows a health professional or user to vary the dose to meet specific therapeutic requirements by manipulating the treatment duration. Alternatively, as shown in FIG. 3D, the dosing can be controlled via programmable multi-burst acoustics with constant or variable amplitude and/or duration. The acoustofluidic patch could deliver a constant volume during each burst and achieve a predictable cumulative dose over time. This feature enables controlled drug release and targeted therapy.

Transdermal delivery of naloxone ex vivo and in vivo. For the hollow needle design, Applicant adapted an oblique cone shape to maximize the hollow bore diameter (0.6 mm) while maintaining the sharpness of the tip. The measured average tip size was 27.9±2.4 μm, approaching the resolution limit of the commercial 3D printer used. Representative hematoxylin and eosin (H&E) staining of the skin cryosection with or without microneedle patch application is shown in FIG. 4A. The microneedle penetrated through the stratum corneum (SC) exposing the epidermis to acoustic streaming. Thus, upon insertion into freshly harvested mouse skin, the microneedle fully penetrates the skin and reaches dermis layer. After the patch treatment, the needle-indents on the skin quickly became almost invisible as the skin started to recover within 6 hours, and no visible irritation was observed after 24 hours.

To validate the patch performance for drug delivery, Applicant first applied rhodamine B (RhB) dye-loaded patch on freshly harvested mouse skin. The test results showed that the patch was able to perform precise dosing by varying treatment time. Based on measuring the change of fluorescent intensity with different durations of acoustic streaming, the delivery speed of the device was ˜11.4 times faster than passive diffusion. Applicant then further verified the delivery efficacy of the patch specifically for naloxone. Freshly harvested mouse skin tissue was treated with a naloxone patch for different durations and immediately frozen for later analysis with liquid nitrogen. The naloxone level was not detectable in the 0 min treatment duration group. By using HPLC-MS to measure naloxone concentration in freshly harvested mouse skin, the mean transdermal delivery rate was 0.55±0.05 mg/min in situ (concentration of naloxone in the patch was 15 mg/ml, FIG. 4B) and could be fine-tuned to achieve different dosing goals.

A positive control group received a subcutaneous injection of 1.5 mg naloxone using a 27 G needle (shown as the needle group). There was no statistically significant difference between the microneedle patch and needle injection. The targeted dose range (1.5 milligrams) is indicated with a red dotted line on FIG. 4C. The naloxone level was not detectable in the negative control (saline). A patch treatment (3 min, 1.5 mg) demonstrated no significant difference compared to subcutaneous injection (1.5 mg), indicating that the patch achieves equivalent performance to the traditional injection method.

To further substantiate this finding, Applicant investigated the biodistribution of naloxone in vivo at different time points following single-dose administration via the patch. The concentration of naloxone in the serum, brainstem and liver were measured with HPLC-MS. Within 5-10 min after the acoustofluidic patch treatment naloxone was readily detectable in all tissues (FIG. 4D). Serum naloxone levels at 5 and 10 minutes were in good agreement with previous reported in vivo studies with parenteral administration of naloxone. Due to its high lipid content, naloxone content was about 5 times higher in the brain stem compared to the liver. These results validated the capability of our patch system to rapidly deliver high concentrations of naloxone to the brain, a crucial factor for therapeutic efficacy.

I claim:

1. An acoustofluidic patch comprising:
a surface acoustic wave (SAW) device;
an acoustofluidic streaming chamber; and
a hollow microneedle.

2. The acoustofluidic patch of claim 1, wherein the SAW device comprises a piezoelectric substrate and at least one pair of interdigital transducers.

3. The acoustofluidic patch of claim 1, wherein the acoustofluidic streaming chamber comprises an inlet, and the hollow microneedle is separably coupled to the inlet.

4. The acoustofluidic patch of claim 1, wherein the SAW device is separatable from the acoustofluidic chamber.

5. The acoustofluidic patch of claim 6, wherein the hollow microneedle has a tip size ranging from about 0.1 mm to about 4 mm.

6. The acoustofluidic patch of claim 1, wherein the orientation angle of the acoustofluidic streaming chamber to the SAW device is 22 to 202 degrees.

7. A transdermal delivery patch comprising the acoustofluidic patch of claim 1 wherein a fluid medium is contained within the acoustic streaming chamber and the fluid has a viscosity from about 0.5 to about 500 cp/mPa's.

8. The transdermal delivery patch of claim 7, wherein the fluid within the acoustofluidic streaming chamber comprises a therapeutic agent.

9. A method for transdermally administering a therapeutic agent to a subject, the method comprising:
applying the patch of claim 8 to the skin of the subject wherein the hollow microneedle penetrates the subject's skin;
activating the SAW device through actuation of an AC signal to generate an acoustic stream within the acoustofluidic chamber and move fluid from the acoustofluidic chamber out the hollow microneedle.

10. The method of claim 9, wherein the AC signal comprises a radio frequency signal.

11. The method of claim 9, wherein the actuating of the AC signal occurs for at least 30 seconds, 1 min, 5 min, 10 min, 15 min, 20 min, 25 min, 30 min, 35 min, 40 min, 45 min, 50 min, 55 min, or 60 min.

12. The method of claim 9, wherein the therapeutic agent has an average flow rate of about 5-60 μL/min, 5-40 μL/min, 5-20 μL/min, 10-50 μL/min, 10-15 μL/min, 15-25 μL/min, 15-55 μL/min, 25-55 μL/min through the hollow microneedle.

13. A biofluid sampling patch comprising the acoustofluidic patch of claim 1 wherein the acoustofluidic streaming chamber contains no fluid prior to use.

14. The biofluid sampling patch of claim 13, further comprising an organic electrochemical transistor.

15. A method for transdermal sampling a biofluid from a subject, the method comprising:
applying the patch of claim 13 to the skin of the subject wherein the hollow microneedle penetrates the subject's skin;
activating the SAW device through actuating an AC signal to generate an acoustic stream within the acoustofluidic chamber and moving fluid into the acoustofluidic chamber through the hollow microneedle.

16. The method of claim 15, wherein the biofluid is blood, interstitial fluid, or a combination thereof.

17. The method of claim 15, wherein the AC signal comprises a radio frequency signal.

18. The method of claim 15, wherein the actuating of the AC signal occurs for at least 30 seconds, 1 min, 5 min, 10 min, 15 min, 20 min, 25 min, 30 min, 35 min, 40 min, 45 min, 50 min, 55 min, or 60 min.

19. The method of claim 15, wherein sampling the biofluid comprises removing a biofluid sample volume from the subject of 0.1-5 μL, 0.1-1 μL, 0.1-0.75 μL, 0.1-0.5 μL, 0.1-0.25 μL, 0.2-0.6 μL, or 0.25-0.5 μL, wherein each range is inclusive.

20. The method of claim 15, wherein the biofluid has a flow rate of 0.5-40 μL/min, 10-20 μL/min, 10-15 μL/min, or 15-20 μL/min from the subject through the hollow needle, the biofluid having a temperature of about 37.5° C.
